(19) United States
(12) Patent Application Publication
(10) Pub. No.: US 2026/0263040 A1
(43) Pub. Date: Sep. 10, 2026

(54) LOW NOISE MOTOR DRIVE UNIT

(71) Applicant: Boston Scientific Scimed, Inc., Maple Grove, MN (US)

(72) Inventors: Donald Masters, Sylmar, CA (US); Corydon Carlson, Stillwater, MN (US); Andrew Brian Graveley, Shoreview, MN (US)

(73) Assignee: Boston Scientific Scimed, Inc., Maple Grove, MN (US)

(21) Appl. No.: 19/555,090

(22) Filed: Mar. 3, 2026

Related U.S. Application Data
(60) Provisional application No. 63/788,251, filed on Apr. 14, 2025.
(60) Provisional application No. 63/766,518, filed on Mar. 4, 2025.

(51) Int. Cl. A61B 8/00; A61B 8/08; A61B 8/12

(52) U.S. Cl. CPC A61B 8/445; A61B 8/0891; A61B 8/12

(57) ABSTRACT

An intravascular ultrasound (IVUS) system includes a catheter having a flexible body with an imaging assembly disposed within the flexible body. An ultrasound transducer is coupled to a distal end region of the imaging assembly. A motor drive unit coupled to a proximal end of the imaging assembly includes a stationary portion with a power source generating an AC power signal and a rotating portion configured to rotate with the imaging assembly. A rotary transformer couples the stationary and rotating portions. The rotating portion includes an amplifier electrically coupled to the ultrasound transducer, a rectifier converting the AC power signal to DC power for the amplifier, and transmit/receive switches protecting the amplifier during transmit mode.

### CROSS-REFERENCE TO RELATED APPLICATIONS

This application claims the benefit of priority under 35 U.S.C. §119 of U.S. Provisional Application No. 63/788,251, filed Apr.14, 2025 & U.S. Provisional Application No. 63/766,518, filed March 4, 2025, the entire disclosure of which is hereby incorporated by reference.

### TECHNICAL FIELD

The present disclosure pertains to medical imaging, and systems and methods for medical imaging. More particularly, the present disclosure pertains to a low noise motor drive unit of intravascular ultrasound catheters.

### BACKGROUND

A wide variety of medical imaging systems and methods have been developed for medical use, and more specifically, for use in imaging vascular anatomy. Some of these systems and methods include intravascular imaging modalities. These systems and methods include various configurations and may operate or be used according to any one of a variety of methods. Of the known vascular imaging systems and methods, each has certain advantages and disadvantages. Accordingly, there is an ongoing need to provide alternative systems and methods for vascular imaging and assessment.

### BRIEF SUMMARY

This disclosure provides design, material, manufacturing method, and use alternatives for medical devices. An intravascular imaging system is disclosed.

In an example, an intravascular ultrasound (IVUS) system may include a catheter, an imaging assembly disposed within the catheter, an ultrasound transducer coupled to a distal end region of the imaging assembly, and a motor drive unit coupled to a proximal end of the imaging assembly, where the motor drive unit may include a stationary portion comprising a power source configured to generate an AC power signal, a rotating portion configured to rotate with the imaging assembly, a rotary assembly coupling the stationary portion to the rotating portion, an amplifier disposed on the rotating portion and electrically coupled to the ultrasound transducer, and a rectifier disposed on the rotating portion and configured to convert the AC power signal from the power source into DC power for powering the amplifier.

Alternatively, or additionally to any of the examples above, in another example, the AC power signal may have a frequency in the range of about 1 kilohertz (kHz) to about 1 megahertz (MHz).

Alternatively, or additionally to any of the examples above, in another example, the system may further include an inductor disposed between the rotary assembly and the rectifier and configured to filter high frequency signals.

Alternatively, or additionally to any of the examples above, in another example, the imaging assembly may be directly coupled to a ground of the amplifier.

Alternatively, or additionally to any of the examples above, in another example, the system may further include transmit/receive switches disposed on an input and output of the amplifier configured to protect the amplifier during transmit mode.

Alternatively, or additionally to any of the examples above, in another example, the system may further include disconnect diodes configured to create a bypass path around the amplifier during transmit mode.

Alternatively, or additionally to any of the examples above, in another example, the transmit/receive switches and the disconnect diodes may be configured to create a bypass circuit around the amplifier during transmit mode.

Alternatively, or additionally to any of the examples above, in another example, the motor drive unit may further include a field programmable gate array (FPGA) and analog-to-digital converter (ADC) configured to digitize signals from the ultrasound transducer.

Alternatively, or additionally to any of the examples above, in another example, the motor drive unit may further include active filtering components.

Alternatively, or additionally to any of the examples above, in another example, the motor drive unit may further include a microcontroller unit configured to control rotation of the imaging assembly.

Alternatively, or additionally to any of the examples above, in another example, signals received from the ultrasound transducer may be configured to pass through the amplifier.

Alternatively, or additionally to any of the examples above, in another example, the motor drive unit may further include a digital output interface configured to transmit digitized signals to an external processing system.

Alternatively, or additionally to any of the examples above, in another example, the rotary assembly may include a brushed metal slip ring and a rotary transformer.

Alternatively, or additionally to any of the examples above, in another example, the rotary assembly may include a liquid metal slip ring.

Alternatively, or additionally to any of the examples above, in another example, the rotary assembly may include rotary transformer having a magnetic coupling.

In an example, an intravascular ultrasound (IVUS) system may include a catheter, an imaging assembly disposed within the catheter, an ultrasound transducer coupled to a distal end region of the imaging assembly, and a motor drive unit coupled to a proximal end of the imaging assembly, where the motor drive unit may include a stationary portion comprising a power source configured to generate an AC power signal, a rotating portion configured to rotate with the imaging assembly, a rotary assembly coupling the stationary portion to the rotating portion, an amplifier disposed on the rotating portion and electrically coupled to the ultrasound transducer, a rectifier disposed on the rotating portion and configured to convert the AC power signal from the power source into DC power for powering the amplifier, and transmit/receive switches disposed on an input and output of the amplifier configured to protect the amplifier during transmit mode.

Alternatively, or additionally to any of the examples above, in another example, the AC power signal may have a frequency in the range of about 1 kilohertz (kHz) to about 1 megahertz (MHz).

Alternatively, or additionally to any of the examples above, in another example, the system may further include an inductor disposed between the rotary transformer and the rectifier configured to filter high frequency signals.

Alternatively, or additionally to any of the examples above, in another example, the imaging assembly may be directly coupled to a ground of the amplifier.

Alternatively, or additionally to any of the examples above, in another example, the system may further include disconnect diodes configured to create a bypass path around the amplifier during transmit mode.

Alternatively, or additionally to any of the examples above, in another example, the motor drive unit may further include a field programmable gate array (FPGA) and analog-to-digital converter (ADC) configured to digitize signals from the ultrasound transducer.

Alternatively, or additionally to any of the examples above, in another example, the motor drive unit may further include active filtering components.

Alternatively, or additionally to any of the examples above, in another example, the motor drive unit may further include a microcontroller unit configured to control rotation of the imaging assembly.

Alternatively, or additionally to any of the examples above, in another example, the ultrasound transducer may include a piezoelectric micromachined ultrasonic transducer (PMUT) or a capacitive micromachined ultrasonic transducer (CMUT).

Alternatively, or additionally to any of the examples above, in another example, signals received from the ultrasound transducer may be configured to pass through the amplifier.

In an example, an intravascular imaging system may include an imaging assembly having a transducer, a motor drive unit coupled to the imaging assembly, where the motor drive unit may include a rotating portion mechanically coupled to the transducer, an amplifier disposed on the rotating portion, a power transmission mechanism configured to provide DC power to the amplifier, and a digitization circuit configured to digitize signals from the transducer before transmission to an external processing system.

Alternatively, or additionally to any of the examples above, in another example, the power transmission mechanism may include a brushed metal slip ring.

Alternatively, or additionally to any of the examples above, in another example, the power transmission mechanism may include a liquid metal slip ring.

Alternatively, or additionally to any of the examples above, in another example, the power transmission mechanism may include a magnetic coupling.

Alternatively, or additionally to any of the examples above, in another example, the digitization circuit may include a field programmable gate array and analog-to-digital converter.

In an example, an intravascular ultrasound imaging system may include an imaging assembly including a drive shaft and an ultrasound transducer disposed adjacent to a distal end of the drive shaft, a motor drive unit having a stationary portion and a rotating portion, the rotating portion mechanically and electrically coupled to the drive shaft, where the motor drive unit may include an amplifier disposed on the rotating portion, a field programmable gate array (FPGA) and analog-to-digital converter (ADC) disposed within the motor drive unit and configured to digitize signals from the ultrasound transducer, a microcontroller unit configured to control rotation of the brushless DC motor and manage memory for the digitized signals, and a digital output interface configured to transmit the digitized signals to an external processing system.

Alternatively, or additionally to any of the examples above, in another example, the system may further include a rectifier disposed on the rotating portion and configured to convert an AC power signal from a power source into DC power for powering the amplifier.

Alternatively, or additionally to any of the examples above, in another example, the system may further include a first transmit/receive switch at an input of the amplifier and a second transmit/receive switch at an output of the amplifier.

Alternatively, or additionally to any of the examples above, in another example, the system may further include a first diode adjacent to the first transmit/receive switch and a second diode adjacent to the second transmit/receive switch.

Alternatively, or additionally to any of the examples above, in another example, the first and second transmit/receive switches and the first and second diodes may be configured to create a bypass circuit around the amplifier during transmit mode.

In an example, an intravascular ultrasound (IVUS) system may include a catheter, an imaging assembly disposed within the catheter, an ultrasound transducer coupled to a distal end region of the imaging assembly, and a motor drive unit coupled to a proximal end of the imaging assembly, where the motor drive unit may include a stationary portion comprising a power source configured to generate a power signal, a rotating portion configured to rotate with the imaging assembly, a first rotating transformer coupling the stationary portion to the rotating portion, a second rotating transformer coupling the stationary portion to the rotating portion, an amplifier disposed on the rotating portion and electrically coupled to the ultrasound transducer, and a rectifier disposed on the rotating portion and configured to convert the power signal from the power source into DC power for powering the amplifier.

Alternatively or additionally to any of the examples above, in another example, the second rotating transformer may include a resonant tank transmitter and a resonant tank receiver.

Alternatively or additionally to any of the examples above, in another example, the second rotating transformer may include a contactless energy transfer mechanism.

Alternatively or additionally to any of the examples above, in another example, the contactless energy transfer mechanism may include a stationary primary winding and a rotating secondary winding.

Alternatively or additionally to any of the examples above, in another example, the contactless energy transfer mechanism may further include a first ferrite core extending into a central aperture of the stationary primary winding and a second ferrite core extending into a central aperture of the rotating secondary winding.

Alternatively or additionally to any of the examples above, in another example, the first ferrite core and the second ferrite core may be separated by an airgap.

Alternatively or additionally to any of the examples above, in another example, the imaging assembly may be directly coupled to a ground of the amplifier.

Alternatively or additionally to any of the examples above, in another example, the first rotating transformer may be configured to transmit radiofrequency signals.

Alternatively or additionally to any of the examples above, in another example, the second rotating transformer may be configured to transmit power.

Alternatively, or additionally to any of the examples above, in another example, the system may further include a low noise voltage regulator disposed between the rotating transformer and the amplifier configured to filter DC signals.

Alternatively, or additionally to any of the examples above, in another example, the system wherein the rectifier is disposed between the rotational transformer and a DC filter.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1A is a partially cut away side view of an example medical device;

FIG. 1B is a side view of the example medical device of FIG. 1A;

FIG. 2 is a perspective view showing the medical device of FIGS. 1A and 1B coupled to a motor drive unit;

FIG. 3 is a partial cross-sectional view of the motor drive unit of FIG. 2 with the medical device disconnected therefrom;

FIG. 4 is a schematic view of some portions of the motor drive unit and the imaging assembly;

FIG. 5 is a schematic view of the motor drive unit incorporating digitization and synchronization circuitry into the motor drive unit;

FIG. 6 is a schematic view of some portions of the motor drive unit and the imaging assembly having an alternative rotary assembly;

FIG. 7 is a partial perspective view of an illustrative contactless energy transfer mechanism; and

FIG. 8 is a schematic view of some portions of the motor drive unit and the imaging assembly having another alternative rotary assembly.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, or characteristics. However, such recitations do not necessarily mean that all embodiments include the particular features, structures, or characteristics. Additionally, when particular features, structures, or characteristics are described in connection with one embodiment, it should be understood that such features, structures, or characteristics may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Rotational intravascular ultrasound (IVUS) may be used for high-resolution imaging of the blood vessels during percutaneous coronary interventions (PCI). Image quality and/or image depth may be limited by the signal-to-noise ratio (SNR) of the IVUS system. One factor limiting the SNR of the system is the susceptibility of the system to electrical noise. The present disclosure is directed towards systems for increasing the SNR of an IVUS system. While the present disclosure is described with respect to intravascular imaging, the devices and methods described herein can be used for pulmonary procedures/imaging or in other anatomy, as desired.

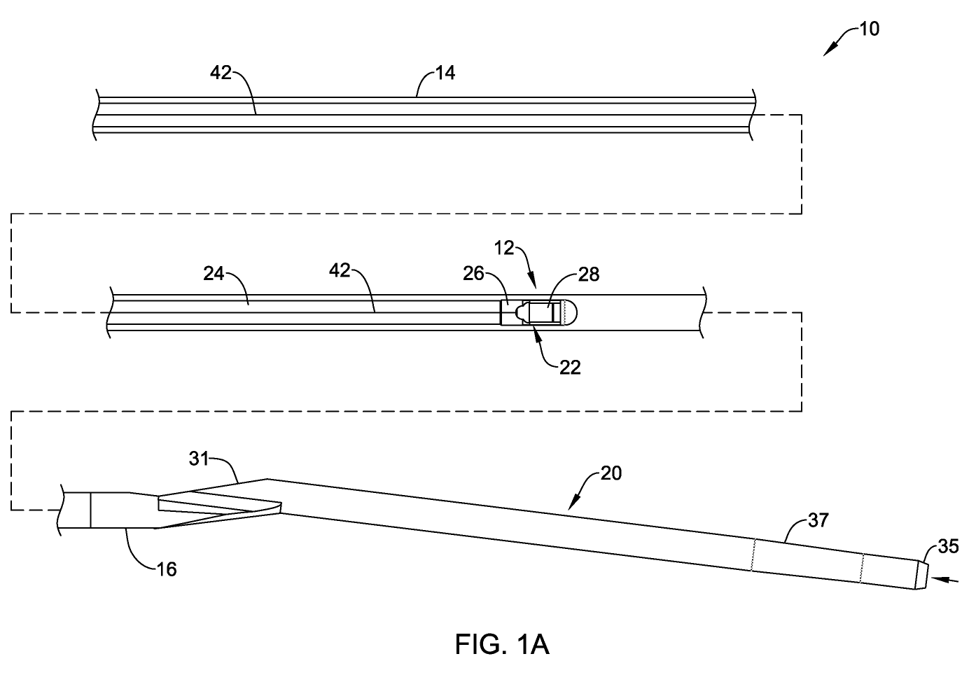

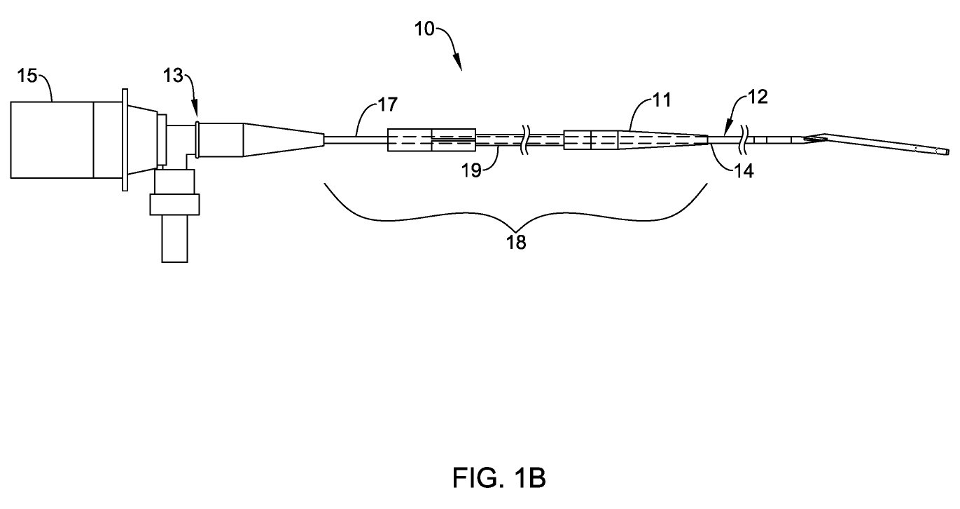

FIG. 1A is a side view of an example medical device 10. In at least some instances, the medical device 10 takes the form of an imaging medical device. For example, the medical device 10 may be an IVUS device that may be used to image a blood vessel. The structure/form of the medical device 10 can vary. In some instances, the medical device 10 may include an elongate shaft 12 having a proximal end region 14 and a distal end region 16. A tip member 20 may be coupled to or otherwise disposed adjacent to the distal end region 16 of the elongate shaft 12. The tip member 20 may include a guidewire lumen 29 having a guidewire exit port 31, an atraumatic distal end 35, one or more radiopaque markers 37, and/or other features. In some embodiments, the tip member 20 may extend at a non-parallel angle to the proximal end region 14 of the elongate shaft 12.

An imaging assembly 22 may be movably disposed within a lumen of the shaft 12. In general, the imaging assembly 22 may be used to capture/generate images of a blood vessel. In some instances, the medical device may include devices and/or features similar to those disclosed in U.S. Patent Application Pub. No. 2012/0059241 and U.S. Patent Application Pub. No. 2017/0164925, the entire disclosures of which are herein incorporated by reference. In at least some instances, the medical device 10 may resemble and/or include features that resemble the OPTICROSS™ Imaging Catheter or the OPTICROSS™ HD Imaging Catheter, commercially available from BOSTON SCIENTIFIC, Marlborough, MA.

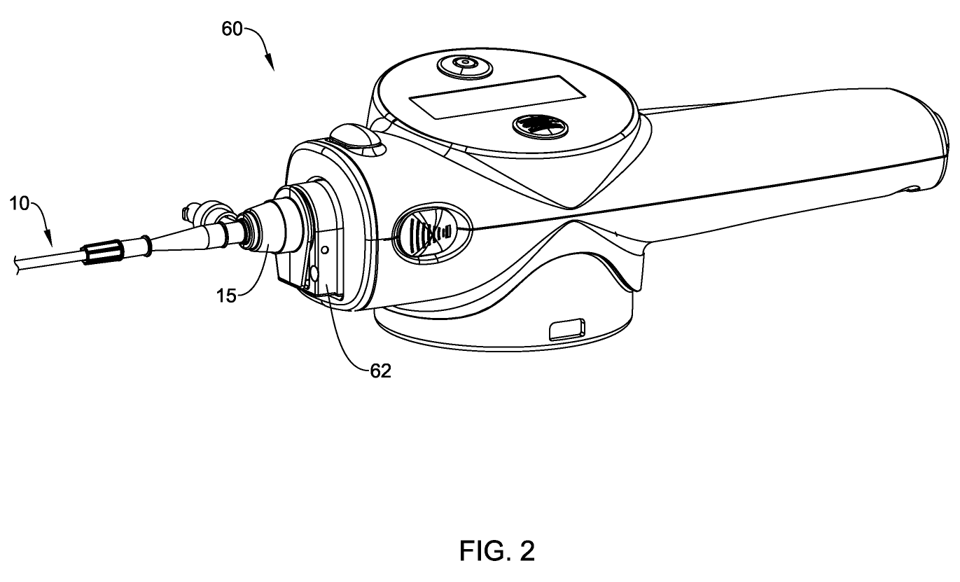

The imaging assembly 22 may include a drive cable or shaft 24, a housing 26, and an imaging member or transducer 28 coupled to the drive cable 24 and/or housing 26. In at least some instances, the transducer 28 includes an ultrasound transducer. In some cases, the transducer 28 may be a piezoelectric micromachined ultrasonic transducer (PMUT) or a capacitive micromachined ultrasonic transducer (CMUT). In other examples, the transducer 28 may be a bulk piezoelectric transducer. In yet other examples, the transducer 28 may be a composite piezoelectric transducer. The transducer 28 may include lead zirconate titanate (PZT), lead-magnesium-niobate lead-titanate (PMN-PT), or other materials, as desired. Other transducers are also contemplated. The transducer 28 may be rotatable and/or axially translatable relative to the shaft 12. In order to do so, the shaft 12 may be connected to a control unit such as a motor drive unit (e.g., a motor drive unit 60 as shown in FIG. 2). Illustrative drive motor units are described in U.S. Patent Application Pub. No. 2024/0325713, the entire disclosure of which is herein incorporated by reference. For example, the drive cable 24 may be rotated and/or translated in order to rotate and/or translate the transducer 28 (and the housing 26). A conductor 42 may be coupled to the transducer 28 and extend proximally therefrom. In some instances, the conductor 42 may take the form of a wire or cable (e.g., a coaxial cable) with suitable electrical conduction properties that allow the conductor 42 to energize the transducer 28. The conductor 42 may extend through a lumen of the drive cable 24.

The proximal end region 14 of the elongate shaft 12 may be coupled to a telescoping assembly 18 as shown in FIG. 1B. In general, the telescoping assembly 18 may be configured to allow a medical device operator to move the drive shaft 24 including the imaging assembly 22 proximally and distally within the catheter (e.g., relative to the elongate shaft 12), without having to move the entire catheter within the patient. This allows the catheter operator to easily change the location of the imaging assembly 22 within the patient. For example, the telescoping assembly 18 may be actuated to change the location of the imaging assembly 22 within the elongate shaft 12. An illustrative telescoping section is described in commonly assigned U.S. Patent Application Pub. No. 2023/0309962, the disclosure of which is herein incorporated by reference.

The proximal end region 14 of the elongate shaft 12 may be coupled to the telescoping assembly 18. For example, the proximal end region 14 of the elongate shaft 12 may be coupled to a distal hub 11 of the telescoping assembly 18. A proximal hub 13 may be coupled to the telescoping assembly 18 (e.g., at the proximal end of the telescoping assembly 18). The drive shaft 24 (see FIG. 1A) may extend through the telescoping assembly 18 and be coupled to and/or otherwise secured to the proximal hub 13. The proximal hub 13 may include a connector assembly 15. In general, the connector assembly 15 may allow the medical device 10 (e.g., the elongate shaft 12) to connect to a control unit (e.g., a motor drive unit and/or the like).

The telescoping assembly 18 may include a first sheath 17 and a second sheath 19. In some instances, the first sheath 17 may be understood to be an inner telescoping tube 17 and the second sheath 19 may be understood to be an outer telescoping tube 19. Generally, the outer telescoping tube 19 may be disposed over the inner telescoping tube 17. The inner telescoping tube 17 may be coupled or otherwise secured to the proximal hub 13. The outer telescoping tube 19 may be coupled or otherwise secured to the distal hub 11. The inner telescoping tube 17 may be axially and/or rotatably moveable relative to the outer telescoping tube 19. Because the drive shaft 24 may be secured to the proximal hub 13 and because the elongate shaft 12 may be secured to the distal hub 26, movement of the proximal hub 13 relative to the distal hub 11 results in movement of the inner telescoping tube 17 and the drive shaft 24 relative to the distal hub 11 and/or the elongate shaft 12.

The connector assembly 15 of the medical device 10 may be connected to a drive motor unit 60 (see, for example, FIG. 2) in a manner that permits the rotation and translation of the imaging assembly 22 as well as the ability to power/energize the transducer 28. FIG. 2 is a perspective view showing the medical device 10 coupled to a motor drive unit 60. For example, the connector assembly 15 may be coupled to a connector receptacle 62 on the motor drive unit 60. When doing so, the connector assembly 15 and the connector receptacle 62 may be configured so that the imaging device 28 may be coupled to the motor drive unit 60 in a manner that permits the rotation and translation of the imaging assembly 22 as well as the ability to power/energize the transducer 28.

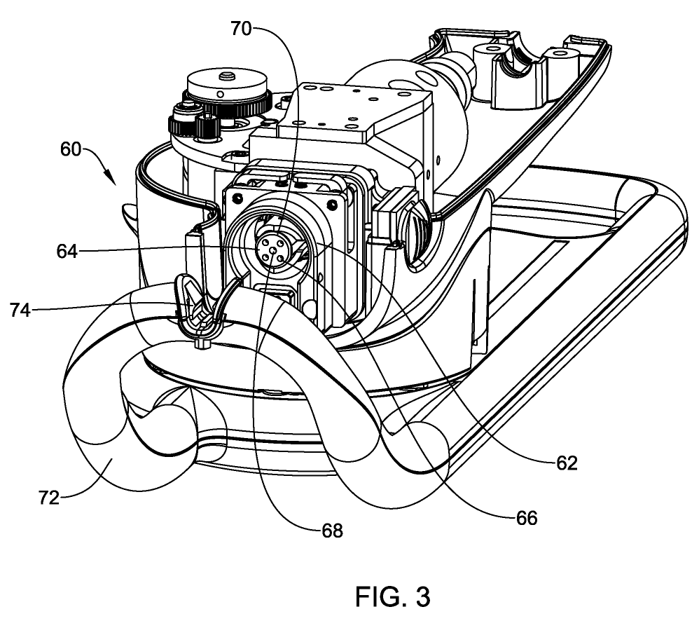

FIG. 3 is a partial cross-sectional view of the motor drive unit 60 with the medical device 10 disconnected therefrom. Here it can be seen that the connector receptacle 62 may include a connector interface 64. In general, the connector receptacle 62 is configured to receive the connector assembly 15 of the medical device 10. The connector interface 64 may be configured to engage with, for example, the electrical and optical connectors that may be part of the connector assembly 15. For example, the connector interface 64 may include a plurality of openings formed therein including one or more electrical pin openings/receptacles 66 and an optical connector opening/receptacle 68. The connector interface 64 may also include an orienting surface 70 (e.g., a cam surface) that is generally designed to help orient the connector assembly 15 in a suitable manner when engaging the connector assembly 15 with the connector receptacle 62. Also shown in FIG. 3 is that the motor drive unit 60 may be coupled to (e.g., slidably coupled to) a translation base or sled 72. The sled 72 may include a catheter cradle region 74 that may be configured to help support the medical device 10 and/or help to keep the elongate shaft 12 stationary during an operational (e.g., pullback) procedure.

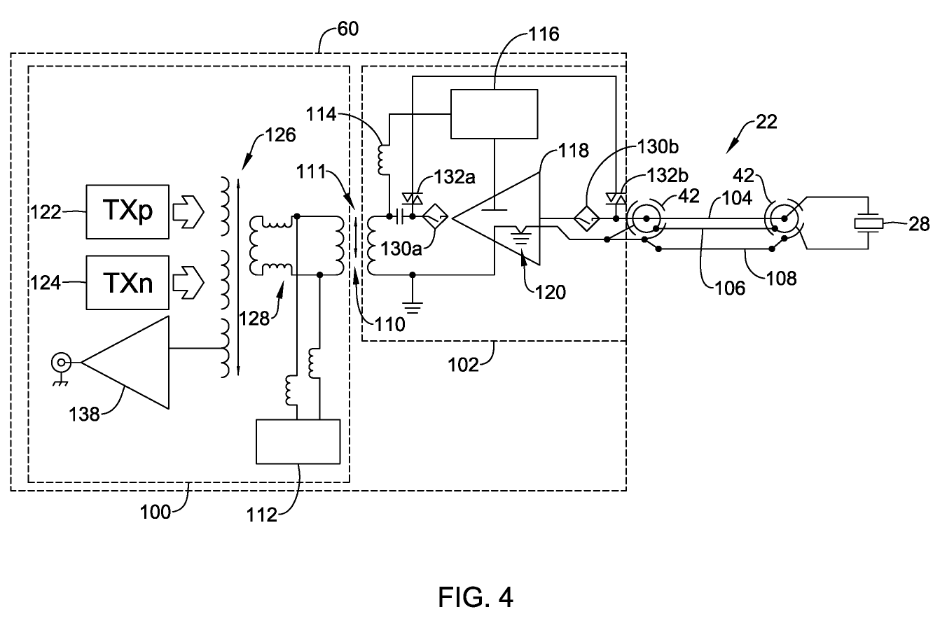

FIG. 4 is a schematic view of some portions of the motor drive unit 60 and the imaging assembly 22. The motor drive unit 60 may include a non-rotating or stationary portion 100 and a rotating portion 102. The imaging assembly 22 is mechanically and electrically coupled to the rotating portion 102 to transfer rotational and axial movement as well as electrical signals between the motor drive unit 60 and the imaging assembly 22. In some configurations, the conductor 42 may be a triaxial cable. For example, the conductor may include a central conductive member 104 configured to be coupled to a first surface of the transducer 28, a first annular conductive member 106 configured to be coupled to a second surface of the transducer 28, and a second annular conductive member 108 configured to be coupled to ground, as will be described in more detail herein. However, the conductor 42 may have other configurations, as desired, such as, but not limited to a coaxial cable, a twisted wire pair, a shielded twisted pair, a flex cable, or the like.

While not explicitly shown, the motor drive unit 60 may include a motor having a motor shaft operably connected to the drive shaft 24 and electronic components (such as, but not limited to, printed circuit boards (PCB), field programmable gate arrays (FPGA), analog to digital converters, filters, time-gain control, transmitter circuitry, receiver circuitry, or the like) for controlling and operating the imaging assembly 22. The stationary portion 100 may include a positive transmit signal line 122 and a negative transmit signal line 124. The positive and negative transmit signal lines 122, 124 may send high voltage transmit signals through the motor drive unit 60 to the imaging assembly 22. The positive and negative transmit signal lines 122, 124 pass through a patient isolation transformer 126. The patient isolation transformer 126 may protect the patient by providing electrical isolation between the patient connected components and the acquisition computing side of the system.

The rotating portion 102 of the motor drive unit 60 may transfer and receive signals (e.g., electrical signals) to and from the stationary portion 100 of the motor drive unit 60. The electrical signals may be passed through a rotary assembly 111 including at least a rotating transformer 110. The rotating transformer 110 may enable electrical signal and power transfer between the stationary electronics of the stationary portion 100 and the rotating components of the rotating portion 102 while maintaining electrical and mechanical isolation. For example, the rotating transformer 110 may allow transmission of both an alternating current (AC) power signal and radiofrequency (RF) signals between the stationary portion 100 and the rotating portion 102 of the motor drive unit 60. The rotating transformer 110 may use electromagnetic induction to transmit power and RF signals between the stationary portion 100 and the rotating portion 102. The AC power may be provided from a power source 112. The power source 112 should provide electrical isolation for patient protection. The AC power may be provided as a low harmonic 1-megahertz (MHz) AC signal. However, the AC power may be provided at other frequencies, as desired. For example, the AC power may have a frequency in the range of about 1 kilohertz (kHz) to about 1 MHz. It is contemplated that a 1 MHz AC signal may be above the main bandwidth of the motor drive unit 60 to avoid interference while being below normal imaging frequencies and the maximum bandwidth capability of the rotating transformer 110.

One or more common mode chokes 128 may be positioned between the patient isolation transformer 126 and a rotating transformer 110 to reduce common mode noise. In some cases, the one or more common mode chokes 128 may be replaced with RLC filters (e.g., circuits including resistors (R), inductors (L) and capacitors (C)) in some applications. RLC filters may be used to filter out unwanted frequencies while allowing others to pass through. It is further contemplated that one or more of the resistors, inductors, or capacitors may be removed or omitted. In some configurations, the patient isolation transformer 126 may be combined with the rotating transformer 110. It is further contemplated that if the rotary assembly 111 is isolated then neither the patient isolation transformer 126 nor the power source 112 have to be isolated. In some instances, if the rotary assembly 111 is isolated, the patient isolation transformer 126 may be eliminated or omitted. In some cases, isolation of the power source 112 may not be required when using high break down wire in the rotating transformer 110.

In some embodiments, the power supply 112 may be a direct current (DC) power supply. In such an embodiment, the electronics coupled to the power source 112 may include a switching regulator controller. A switching regulator controller is an integrated circuit (IC) that controls the timing of a power transistor's switching within a switching regulator, ensuring a stable output voltage by monitoring and adjusting the switching frequency or pulse width. In some cases, the switching regulator controller may be designed to reduce conducted and radiated electromagnetic interference. An illustrative controller may be a LT1683 manufactured by Analog Devices, Inc. (ADI) (Wilmington, MA). The switching regulator controller may enable independent control of voltage and current slew rates of external N-channel MOSFET switches to optimize harmonic content versus efficiency. This configuration can reduce high frequency harmonic power by up to 40 decibels (dB) while maintaining minimal efficiency losses. Some switching regulator controllers may have a low-noise switching regulator design without requiring extensive pre and post regulator filtering or precise synchronization schemes.

The magnetic coupling may allow for power transfer to the rotating components without physical electrical connections. Further, the magnetic coupling may allow for RF signal transmission while maintaining electrical isolation as well as reduced susceptibility to external noise (relative to direct electrical connections). In some configurations, the rotary assembly 111 may include a brushed metal slip-ring for the transmission of power while using a rotational transformer 110 for transmitting the RF signal. A brushed metal slip-ring may include a rotating portion, or rotor, including one or more conductive rings which rotate with the rotating portion and a stationary portion, or stator. The stator may be formed from conductive materials configured to press against the surface or conductive ring(s) of the rotor to maintain a continuous electrical connection. It is contemplated that when the slip-ring is used for only the transmission of power, the rotor of the slip-ring may include two conductive rings. In yet other configurations, the rotary assembly 111 may include a liquid slip-ring for both power transmission and for transmitting the RF signals. A liquid slip-ring may include a rotating portion, or rotor, including one or more conductive rings which rotate with the rotating portion and a stationary portion, or stator. A pool of liquid, such as, but not limited to, mercury or gallium alloy, may be used maintain contact between the rotating and stationary portions. Said differently, the liquid creates a low-resistance path for the electrical signals as the stator does not directly contact the rotor. It is contemplated that when the slip-ring is used for both the transmission of power and RF signals, the rotor of the slip-ring may include four conductive rings. It is contemplated a second magnetic coupling, brushed metal slip ring, or liquid slip-ring may require a DC-DC isolator to provide electrical isolation between the stationary portion 100 and the rotating portion 102 of the system while converting DC power.

The AC signal may travel across the rotating transformer 110 to the rotating portion 102. The AC signal may be super positioned on the high frequency RF signals provided to the transducer 28. At the rotating portion 102, the AC signal may be rectified to direct current (DC) voltage at an AC/DC rectifier 116. In some cases, the AC signal may be rectified to 5 or 3.3 volts (V) DC. However, the AC signal may be rectified to other DC voltages, as desired. The DC voltage may be used to power an amplifier 118 on the rotating portion 102. An inductor 114 may be positioned at the input of the AC/DC rectifier 116. The inductor 114 may function as a low-pass filter to prevent interference between the power transmission and the higher frequency transmit/receive signals. Said differently, the inductor 114 may prevent the power system (e.g., voltage supplying the amplifier 118) from interfering with the RF signals transmitted to the transducer 28.

The amplifier 118 may be configured to reduce the susceptibility of the received electrical signal to noise thus improving the SNR. For example, the drive cable 24 may be connected directly to the ground 120 of the amplifier 118 (e.g., via the second annular conductive member 108). The electrical signal is amplified before passing through the rotating transformer 110 and other transformers of the motor drive unit 60. This may reduce the susceptibility of the received electrical signal to external noise which may be induced on the outside of the drive cable 24 and/or conductor 42. It is contemplated that there may be a low resistance connection between the ground 120 of the amplifier circuit and the drive cable 24 as well as a low resistance connection between the drive cable 24 and the negative side of the transducer 28. Illustrative low resistance connections are described in commonly assigned U.S. Patent Application Number 63/718,037, the disclosure of which is hereby incorporated by reference.

The amplifier 118 may have a lower noise figure such that the amplifier 118 introduces less electronic noise into the signal during the amplification process. The lower noise figure amplifier 118 may help improve the overall system performance by minimizing the amount of noise added to the received signal during amplification. It is contemplated that amplifying the received signal prior to transmitting the signal across the rotating transformer 110 may increase the signal relative to the noise floor. For example, transformers (e.g., rotating transformer 110, and the like) and/or common mode chokes may cause some signal loss or degradation as signals pass through them. Amplification of the received electrical signal before it experiences losses through the transformers 110, common mode chokes, or the like increases the signal strength relative to the noise floor thereby improving the overall SNR. Improving the SNR may enable better image quality, greater imaging depth, and/or automated lumen analysis. It is further contemplated that increasing the SNR may allow for imaging at higher frequencies and/or higher resolution at the same imaging depth. The amplifier 118 may also reduce the need for gain in an amplifier 138 on the isolation transformer 126. Lowering the gain of the amplifier 132 in the stationary portion 100 may reduce the amount of noise that will be picked up for electronics inside of the stationary portion 100 of the motor drive unit 60.

The power delivery system of the motor drive unit 60 for supplying power to the amplifier 118 may work alongside transmit/receive (T/R) switches 130a, 130b as well as disconnect diodes 132a, 132b adjacent to the T/R switches 130a, 130b. The T/R switches 130a,130b and the diodes 132a, 132b may create a circuit around the amplifier 118 during transmit mode. For example, providing T/R switches 130a, 130b on both the input and the output of the amplifier 118 may provide time-division-multiplexing between transmit/receive modes while providing overvoltage protection of the input and output of the amplifier 118 from high voltage transmit signals. When in transmit mode, the diodes 132a, 132b conduct and create a bypass path around the amplifier 118, effectively disconnecting the amplifier 118 from the circuit. When in receive mode, the diodes 132a, 132b are non-conducting, allowing the received signals to pass through the amplifier 118 normally. This arrangement may protect the amplifier 118 during high-voltage transmit operations while still allowing the amplifier 118 to amplify the much smaller received signals during receive mode. Said differently, the T/R switches 130a, 130b may provide temporal isolation between high-voltage transmit signals and sensitive receive amplification.

Generally, the receive signal is generated at the transducer 28 and travels through the conductor 42 to the motor drive unit 60. At this stage, external electrical noise may be induced on the outside of the drive cable 24 and/or conductor 42. The received signal may enter the low-noise amplifier 118 positioned within the rotating portion 102 of the motor drive unit 60. The conductor 42 is directly connected to the amplifier ground 120 which may reduce susceptibility to external noise. The receive signal is amplified (at the amplifier 118) prior to passing through the rotating transformer 110 and the patient isolation transformer 126. This may increase signal strength relative to the noise floor thus increasing the SNR. The signal may then be transferred to an external system for processing. For example, the signal may be transferred over a cable to an external computing system for processing.

In some configurations, the amplifier 118 may be provided at the imaging assembly 22. An amplifier 118 placed in the distal end of the imaging assembly 22 may use an application specific integrated circuit (ASIC), in order to fit within the tip of the imaging assembly 22. An ASIC may be suited for use with a CMUT or PMUT transducer that may be easier to electrically connect to or even manufactured on the same wafer die. In some examples, an amplifier 118 placed in the imaging assembly 22 may be used for a separate receiving transducer for a different mode of imaging. Some applications may include but are not limited to, photoacoustic imaging, contrast harmonic imaging transducers, or tissue-harmonic imaging. In contrast harmonic imaging transducers or tissue-harmonic imaging, the receive transducer may be configured to receive a first or second harmonic of the center frequency of a transmit transducer. A separate receiving transducer may use a separate coaxial cable or electrical connection, and thus not need over-voltage protection.

Additionally, or alternatively, the IVUS system may include separate amplifiers to cover different devices or different transducers within a device. The separate amplifiers may be configured to cover devices with similar or differing bandwidths, as desired.

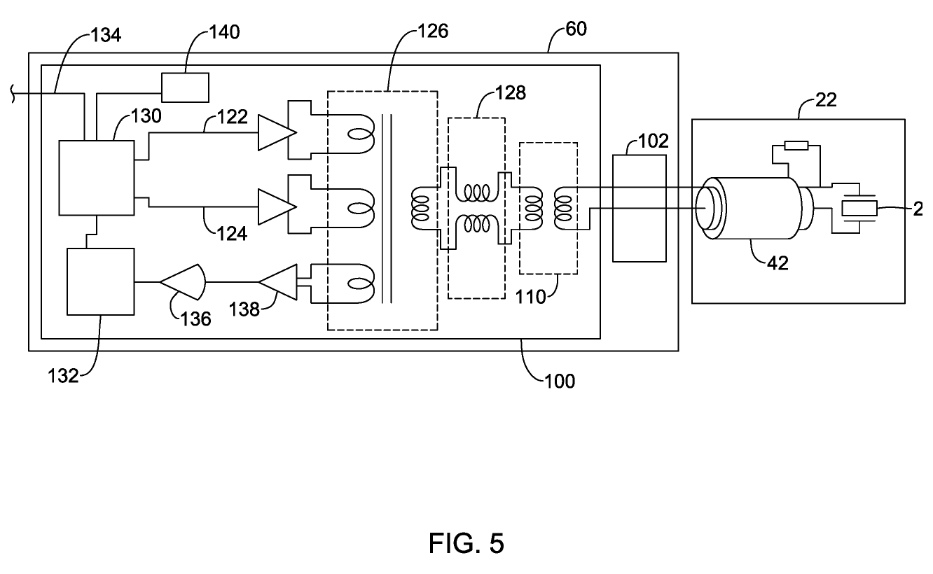

In some configurations, the received electrical signal may be digitized prior to being transmitted to an external system. FIG. 5 is a schematic view of the motor drive unit 60 incorporating digitization and synchronization circuitry into the motor drive unit 60. For brevity, the components of the rotating portion 102 of the motor drive unit 60 are not repeated in FIG. 5. However, it should be understood that the rotating portion 102 of the motor drive unit 60 may include the low noise amplifier 118, rectifier 116, T/R switches 130a, 130b, and diodes 132a, 132b described herein to increase the SNR of the received electrical signal.

The stationary portion 100 of the motor drive unit 60 may include a field-programmable gate array (FPGA) 130 configured to perform digital signal processing and synchronization of the received electrical signals (e.g., from the transducer 28) before transmission to an external processing device (not explicitly shown). The stationary portion 100 of the motor drive unit 60 may also include an analog to digital converter (ADC) 132. Collectively, the FPGA 130 and the ADC 132 may digitize the received electrical signal before traveling over a cable 134 to the external processing system. This may help reduce or eliminate signal susceptibility issues over the cable 134. The cable 134 may have a length in the range of about 12 feet (3.66 meters). Reducing and/or eliminating signal susceptibility issues over the cable 134 may enable the use of a larger motor with a nosier drive circuit such as a brushless-DC motor or a stepper motor with more power and control. Such motors may enable more precise control to reduce non-uniform rotational distortion (NURD) issues, reduce catheter or imaging assembly 22 failure modes by more accurately monitoring and limiting torque, and/or enable more compact or complex designs of the motor drive unit 60.

It is further contemplated that increasing the SNR may allow for some filtering of the signal to be removed which may increase the bandwidth of the IVUS system. Increasing the bandwidth may allow for better resolution in the system. It is further contemplated that increasing the SNR may also allow for the use of active filtering instead of passive filtering which may improve the filtering of the signal. A filter/time-gain control (TGC) 136 may be provided on the stationary portion 100 of the motor drive unit 60. The filter/TGC 136 may process signals between the ADC 132 and an amplifier 138. The filter/TGC 136 may help control and adjust signal gain over time as part of the signal processing chain in the motor drive unit 60 before the signals are digitized.

The stationary portion 100 of the motor drive unit 60 may further include a microcontroller unit 140. The microcontroller unit 140 may be connected to the FPGA 130 and drive circuits for the motors (not explicitly shown) used for rotation and longitudinal positioning of the medical device 10 or components thereof. Furthermore, the microcontroller unit 140 may also be connected to a user interface (not explicitly shown) on the motor drive unit 60. The microcontroller unit 140 may have an increased throughput relative to a peripheral interface controller (PIC) microcontroller unit. The microcontroller unit 140 may be configured to control features such as, but limited to, imaging assembly 22 rotation motor control, memory management for received data, or the like. This may free up surface area at the FPGA 130 to add signal processing features.

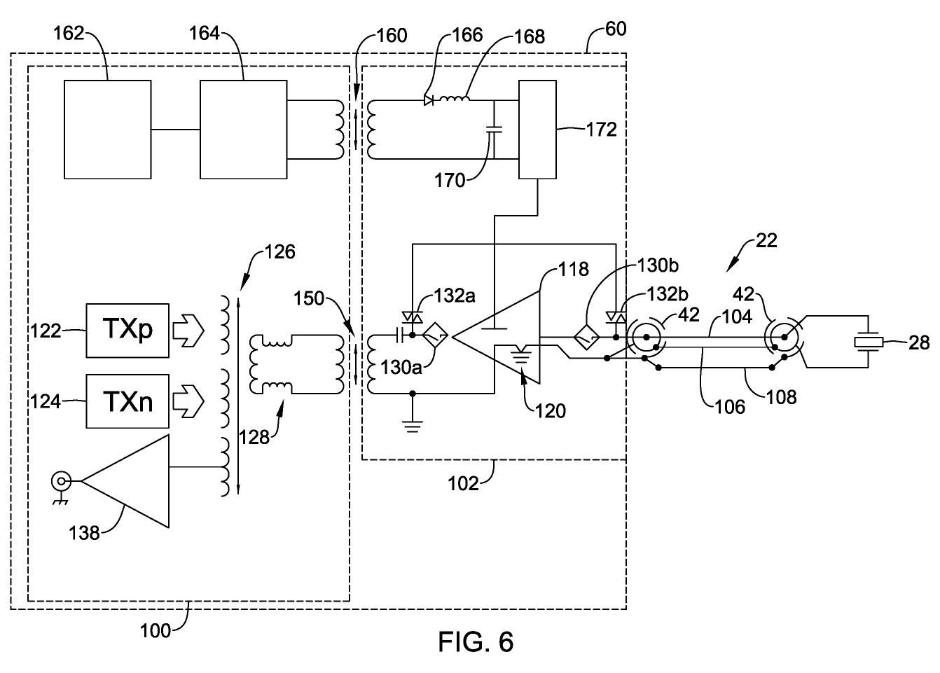

FIG. 6 is a schematic view of some portions of the motor drive unit 60 and the imaging assembly 22 having two rotating transformers 150, 162. For brevity and ease of understanding components that are the same in FIGS. 4 and 6 are identified with like numbers and function in a manner as described with respect to FIG. 4. Generally, a first rotating transformer 150 may transfer RF signals between the stationary portion 100 and the rotating portion 102 using electromagnetic induction.

A second rotating transformer 160 may transmit power to power the amplifier 118 between the stationary portion 100 and the rotating portion 102. In some configurations, a DC-DC power supply 162 may supply power to a DC-AC power supply 164. The DC-AC power supply 64 may supply power through the rotating transformer 160. In some cases, the DC-DC power source 162 may be electrically isolated to provide electrical isolation for patient protection. The AC power (e.g., from the DC-AC power supply 164) may be provided as a low harmonic 1-megahertz (MHz) AC signal. However, the AC power may be provided at other frequencies, as desired. For example, the AC power may have a frequency in the range of about 1 kilohertz (kHz) to about 1 MHz.

The AC signal may travel across the rotating transformer 160 to the rotating portion 102. At the rotating portion 102, the AC signal may be rectified to direct current (DC) voltage at an AC/DC rectifier 166. In some cases, the AC signal may be rectified to 5 or 3.3 volts (V) DC. However, the AC signal may be rectified to other DC voltages, as desired. The DC voltage may be used to power an amplifier 118 on the rotating portion 102. An inductor 168 may be positioned at the output of the AC/DC rectifier 166 to smooth the DC output. A filter capacitor 170 may help reduce noise in the signal. A low noise voltage regulator 172 may be provide a clean, stable, low-noise power supply to the amplifier 118

The amplifier 118 may be configured to reduce the susceptibility of the received electrical signal to noise thus improving the SNR. For example, the drive cable 24 may be connected directly to the ground 120 of the amplifier 118 (e.g., via the second annular conductive member 108). The electrical signal is amplified before passing through the rotating transformer 110 and other transformers of the motor drive unit 60. This may reduce the susceptibility of the received electrical signal to external noise which may be induced on the outside of the drive cable 24 and/or conductor 42. It is contemplated that there may be a low resistance connection between the ground 120 of the amplifier circuit and the drive cable 24 as well as a low resistance connection between the drive cable 24 and the negative side of the transducer 28.

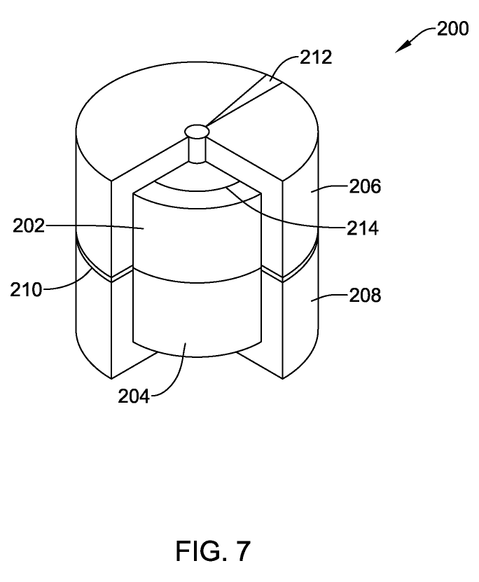

In some configurations, the rotating transformer 160 may include a contactless energy transfer mechanism to transfer energy from the stationary portion 100 to the rotating portion 102. For example, the motor drive unit 60 may employ contactless energy transfer (CET) using adjacent toroidal members with ferrite cores. FIG. 7 is a partial perspective view of an illustrative contactless energy transfer mechanism 200. The contactless energy transfer mechanism 200 may be an alternative to the rotary transformer 160 described herein. For brevity and ease of understanding the remaining components of the motor drive unit 60 and imaging assembly are not shown in FIG. 7. The contactless energy transfer mechanism 200 may provide an alternative to traditional slip-rings, eliminating contact wear and maintenance requirements.

The contactless energy transfer mechanism 200 may include a primary winding 202 configured to be positioned within the stationary portion 100 of the motor drive unit 60 and a secondary winding 204 configured to be positioned within the rotating portion 102 of the motor drive unit 60. The primary winding 202 may have a generally toroidal shape having a central aperture 214 extending through a thickness thereof. Similarly, the secondary winding 204 may have a generally toroidal shape having a central aperture (not explicitly shown) extending through a thickness thereof. A first ferrite core 206 may extend around an outer perimeter of the primary winding 202 and extend into the central aperture 214. A second ferrite core 208 may extend around an outer perimeter of the secondary winding 204 and extend into the central aperture thereof.

In FIG. 7 a portion of the first ferrite core 206 and a portion of the second ferrite core 208 are cut away to more particularly illustrate the primary and secondary windings 202, 204. However, it should be understood the first and second ferrite cores 206, 208 extend about an entirety of the circumference of the primary and secondary windings 202, 204. The contactless energy transfer mechanism 200 may further include an airgap 210 between the first and second ferrite cores 206, 208. The first and second ferrite cores 206, 208 may enhance power transfer capability across the airgap 210 between the rotating secondary winding 204 and the stationary primary winding 202. In some embodiments, the first ferrite core 206 may include an indentation 212 for aligning the contactless energy transfer mechanism 200. However, this is not required. Further, while not explicitly shown, the contactless energy transfer mechanism 200 may include a housing to maintain alignment of the primary winding 202, the secondary winding 204, the first ferrite core 206, and the second ferrite core 208.

The airgap 210 may space the stationary primary winding 202 and the stationary first ferrite core 206 from the rotating secondary winding 204 and the rotating second ferrite core 208. The airgap 210 may be in the range of about 1 millimeter to about 5 millimeters. This may eliminate contact wear since there are no physical electrical connections. It is further contemplated that the lack of contact wear may reduce or eliminate dust particle generation from friction. The contactless energy transfer mechanism 200 may use ferrite core materials specifically chosen to optimize the magnetic coupling and power transfer efficiency across the airgap 210. This may allow the contactless energy transfer mechanism 200 to achieve effective power transmission while maintaining electrical isolation between the stationary and rotating portions of the contactless energy transfer mechanism 200. In some cases, the contactless energy transfer mechanism 200 may provide electrical isolation for patient safety. In such an instance, the DC-DC power supply 162 may be omitted.

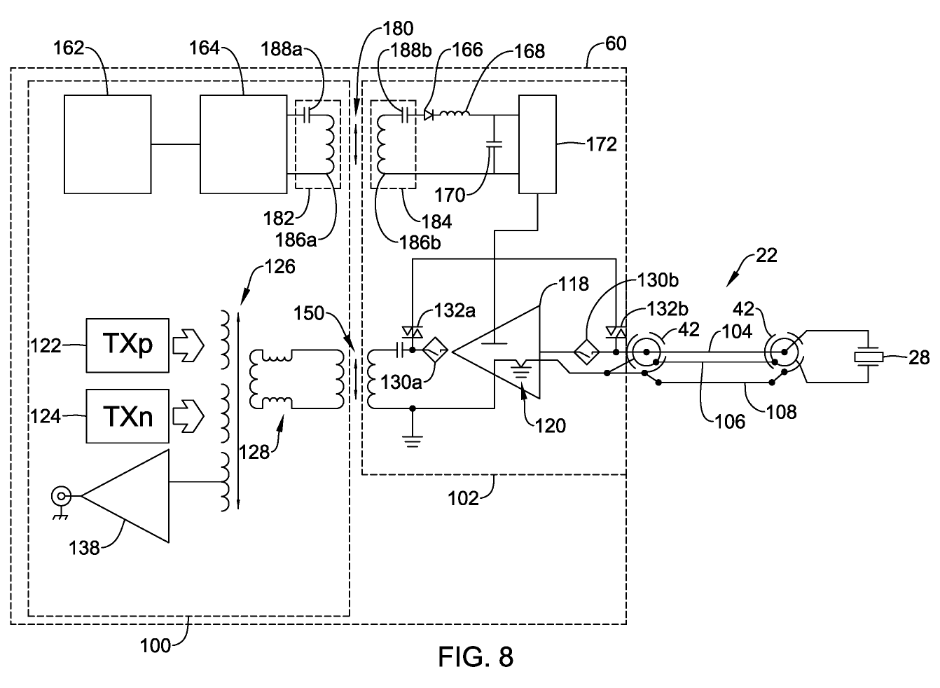

FIG. 8 is a schematic view of some portions of the motor drive unit 60 and the imaging assembly 22 having an alternative power transmission configuration. For brevity and ease of understanding components that are the same in FIGS. 4 and 8 are identified with like numbers and function in a manner as described with respect to FIG. 4. Generally, a first rotating transformer 150 may transfer RF signals between the stationary portion 100 and the rotating portion 102 using electromagnetic induction.

A second rotating transformer 180 may transmit power to power the amplifier 118 between the stationary portion 100 and the rotating portion 102. In some configurations, a DC-DC power supply 162 may supply power to a DC-AC power supply 164. The DC-AC power supply 64 may supply power through the rotating transformer 180. In some cases, the DC-DC power source 162 may be electrically isolated to provide electrical isolation for patient protection. The AC power (e.g., from the DC-AC power supply 164) may be provided as a low harmonic 1-megahertz (MHz) AC signal. However, the AC power may be provided at other frequencies, as desired. For example, the AC power may have a frequency in the range of about 1 kilohertz (kHz) to about 1 MHz.

The AC signal may travel across the rotating transformer 180 to the rotating portion 102. The rotating transformer 180 may utilize wireless power transfer through inductive coupling between a resonant tank transmitter 182 and a resonant tank receiver 184. The resonant tank transmitter 182 and the resonant tank receiver 184 may be resonant circuits (e.g., each including an inductor 186a, 186b and a capacitor 188a, 188b) operating at matched frequencies with high Q factors. When the resonant circuits 182, 184 are brought into close proximity within the near field area, evanescent wave coupling may enable highly efficient energy transfer between the resonant tank transmitter 182 and the resonant tank receiver 184. In some cases, isolation of the power source 112 may not be required when using high break down wire in the resonant tank transmitter 182 and the resonant tank receiver 184.

The wireless power transmission path may include four main components: the DC-AC power supply 164 functioning as an inverter on the stationary portion 100, the resonant tank transmitter 182, the resonant tank receiver 184, and a rectifier 166 on the rotating portion 102. The DC-AC power supply 184 converts input DC voltage from the DC-DC power supply 162 to alternating current, generating an alternating field in the resonant tank transmitter 182. Through counter-induction between the resonant tank transmitter 182 and the resonant tank receiver 184, energy transfers between the resonant tank transmitter 182 and resonant tank receiver 184. Following Faraday's law of induction, the alternating current in the resonant tank transmitter 182 induces an alternating voltage in the resonant tank receiver 184, which is then rectified at the rectifier 166. In some cases, the resonant tank transmitter 182 and the resonant tank receiver 184 may provide electrical isolation for patient safety. In such an instance, the DC-DC power supply 162 may be omitted.

At the rotating portion 102, the AC signal may be rectified to direct current (DC) voltage at the AC/DC rectifier 166. In some cases, the AC signal may be rectified to 5 or 3.3 volts (V) DC. However, the AC signal may be rectified to other DC voltages, as desired. The DC voltage may be used to power an amplifier 118 on the rotating portion 102. An inductor 168 may be positioned at the output of the AC/DC rectifier 166 to smooth the DC output. A filter capacitor 170 may help reduce noise in the signal. A low noise voltage regulator 172 may be provide a clean, stable, low-noise power supply to the amplifier 118

The amplifier 118 may be configured to reduce the susceptibility of the received electrical signal to noise thus improving the SNR. For example, the drive cable 24 may be connected directly to the ground 120 of the amplifier 118 (e.g., via the second annular conductive member 108). The electrical signal is amplified before passing through the rotating transformer 110 and other transformers of the motor drive unit 60. This may reduce the susceptibility of the received electrical signal to external noise which may be induced on the outside of the drive cable 24 and/or conductor 42. It is contemplated that there may be a low resistance connection between the ground 120 of the amplifier circuit and the drive cable 24 as well as a low resistance connection between the drive cable 24 and the negative side of the transducer 28.

The materials that can be used for the various components of the system 10 (and/or other systems disclosed herein) may include those commonly associated with medical devices. For simplicity purposes, the following discussion makes reference to the shaft 12 and other components of the system 10. However, this is not intended to limit the devices and methods described herein, as the discussion may be applied to other similar tubular members and/or components of tubular members or devices disclosed herein.

The shaft 12 and/or other components of the system 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as VESTAMID®, GRILAMID® available from EMS American Grilon, and/or the like), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some cases, the shaft 12 and/or other components of the system 10 may include polymeric coatings for fillers. Some examples for suitable polymers for coating or fillers may include, but are not limited to, parylene (poly-para-xylylene), poly-dimethyl siloxane (PDMS), poly-methyl methacrylate (PMMA), and poly-(vinylidene fluoride) (PVDF), polyacrylonitrile (PAN), epoxy resins, or the like.

Adhesives or electrically conductive adhesives may be used in the coupling of various components of the shaft and/or other components of the system 10. Electrically conductive adhesives may include a conductive component, such as, but not limited to iron, silver, copper, nickel, graphite, or the like, suspended in an adhesive. Some examples of adhesives include acrylics, epoxies, urethanes, hydrocolloids, hydrogels, cyanoacrylates, silicones, or the like.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the system 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the system 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system 10 to achieve the same result.

In some embodiments, a degree of Magnetic Resonance Imaging (MRI) compatibility is imparted into the system 10. For example, the system 10, or portions thereof, may be made of a material that does not substantially distort the image and create substantial artifacts (e.g., gaps in the image). Certain ferromagnetic materials, for example, may not be suitable because they may create artifacts in an MRI image. The system 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An intravascular ultrasound (IVUS) system, comprising:
a catheter;
an imaging assembly disposed within the catheter;
an ultrasound transducer coupled to a distal end region of the imaging assembly;
a motor drive unit coupled to a proximal end of the imaging assembly, the motor drive unit comprising:
a stationary portion comprising a power source configured to generate an AC power signal;
a rotating portion configured to rotate with the imaging assembly;
a rotary assembly coupling the stationary portion to the rotating portion;
an amplifier disposed on the rotating portion and electrically coupled to the ultrasound transducer;
a rectifier disposed on the rotating portion and configured to convert the AC power signal from the power source into DC power for powering the amplifier; and
transmit/receive switches disposed on an input and output of the amplifier configured to protect the amplifier during transmit mode.

2. The system of claim 1, wherein the AC power signal has a frequency in the range of approximately 1 kilohertz (kHz) to 1 megahertz (MHz).

3. The system of claim 1, further comprising an inductor disposed between the rotary transformer and the rectifier configured to filter high frequency signals.

4. The system of claim 1, wherein the imaging assembly is directly coupled to a ground of the amplifier.

5. The system of claim 1, further comprising disconnect diodes configured to create a bypass path around the amplifier during transmit mode.

6. The system of claim 1, wherein the motor drive unit further comprises a field programmable gate array (FPGA) and analog-to-digital converter (ADC) configured to digitize signals from the ultrasound transducer.

7. The system of claim 1, wherein the motor drive unit further comprises active filtering components.

8. The system of claim 1, wherein the motor drive unit further comprises a microcontroller unit configured to control rotation of the imaging assembly.

9. The system of claim 1, wherein signals received from the ultrasound transducer are configured to pass through the amplifier.

10. An intravascular ultrasound imaging system, comprising:
an imaging assembly including a drive shaft and an ultrasound transducer disposed adjacent to a distal end of the drive shaft;
a motor drive unit having a stationary portion and a rotating portion, the rotating portion mechanically and electrically coupled to the drive shaft, the motor drive unit comprising:
an amplifier disposed on the rotating portion;
a field programmable gate array (FPGA) and analog-to-digital converter (ADC) disposed within the motor drive unit and configured to digitize signals from the ultrasound transducer;
a microcontroller unit configured to control rotation of the brushless DC motor and manage memory for the digitized signals; and
a digital output interface configured to transmit the digitized signals to an external processing system.

11. The system of claim 10, further comprising a rectifier disposed on the rotating portion and configured to convert an AC power signal from a power source into DC power for powering the amplifier.

12. The system of claim 10, further comprising a first transmit/receive switch at an input of the amplifier and a second transmit/receive switch at an output of the amplifier.

13. The system of claim 12, further comprising a first diode adjacent to the first transmit/receive switch and a second diode adjacent to the second transmit/receive switch.

14. An intravascular ultrasound (IVUS) system, comprising:
a catheter;
an imaging assembly disposed within the catheter;
an ultrasound transducer coupled to a distal end region of the imaging assembly;
a motor drive unit coupled to a proximal end of the imaging assembly, the motor drive unit comprising:
a stationary portion comprising a power source configured to generate a power signal;
a rotating portion configured to rotate with the imaging assembly;
a first rotating transformer coupling the stationary portion to the rotating portion;
a second rotating transformer coupling the stationary portion to the rotating portion
an amplifier disposed on the rotating portion and electrically coupled to the ultrasound transducer; and
a rectifier disposed on the rotating portion and configured to convert the power signal from the power source into DC power for powering the amplifier.

15. The system of claim 14, wherein the second rotating transformer comprises a resonant tank transmitter and a resonant tank receiver.

16. The system of claim 14, wherein the second rotating transformer comprises a contactless energy transfer mechanism.

17. The system of claim 16, wherein the contactless energy transfer mechanism comprises a stationary primary winding and a rotating secondary winding.

18. The system of claim 17, wherein the contactless energy transfer mechanism further comprises a first ferrite core extending into a central aperture of the stationary primary winding and a second ferrite core extending into a central aperture of the rotating secondary winding.

19. The system of claim 14, wherein the first rotating transformer is configured to transmit radiofrequency signals.

20. The system of claim 14, wherein the second rotating transformer is configured to transmit power.
